(19) United States
(12) Patent Application Publication
(10) Pub. No.: US 2009/0319031 A1
(43) Pub. Date: Dec. 24, 2009

(54) Bioabsorbable Polymeric Stent With Improved Structural And Molecular Weight Integrity

(76) Inventors: Yunbing Wang, Sunnyvale, CA (US); Stephen D. Pacetti, San Jose, CA (US); Carina V. Harold, Pleasanton, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/142,281

(22) Filed: Jun. 19, 2008

(51) Int. Cl. A61F 2/82; H01J 37/30

(52) U.S. Cl. 623/1.38; 623/1.15; 623/1.49; 264/470; 523/113

(57) ABSTRACT

Various embodiments of the present invention include implantable medical devices such as stents manufactured from polymers, and more particularly, biodegradable polymers including biodegradable polyesters. Other embodiments include methods of fabricating implantable medical devices from polymers. The devices and methods utilize one or more stabilizers, where each stabilizer may be chosen from the following categories: free radical scavengers, peroxide decomposers, catalyst deactivators, water scavengers, and metal scavengers.

### BACKGROUND OF THE INVENTION

1. Field of the Invention

This invention relates to methods of manufacturing polymeric stents.

2. Description of the State of the Art

This invention relates to implantable medical devices, and more particularly, to radially expandable endoprostheses, that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open, and sometimes expand, a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of scaffolding that physically holds open and, if desired, expands the wall of a passageway. Typically, stents are capable of being compressed or crimped onto a catheter so that they can be delivered to, and deployed at, a treatment site. Delivery includes inserting the stent through small lumens using a catheter and transporting it to the treatment site. Deployment includes expanding the stent to a larger diameter once it is at the desired location.

The stent must be able to satisfy a number of mechanical requirements. A stent must possess adequate radial strength which is due to strength and rigidity around a circumferential direction of the stent. In addition, the stent must possess sufficient flexibility to allow for crimping, expansion, and cyclic loading.

Some treatments with implantable medical devices require the presence of the device only for a limited period of time. Once treatment is complete, which may include structural tissue support and/or drug delivery, it may be allowed to remain in the vessel or it may be removed. Alternatively, the device stent may be fabricated from, in whole or in part, of materials that erode or disintegrate through exposure to conditions within the body. Stents fabricated from biodegradable, bioabsorbable, and/or bioerodable materials such as bioabsorbable polymers can be designed to completely erode only after the clinical need for them has ended.

However, there are potential shortcomings in the use of polymers as a material for implantable medical devices, such as stents. Polymers that biodegrade in the body may also degrade during the process of manufacturing the implantable medical device such as a stent. Note that degradation during processing could occur for biostable and biodegradable polymers. The mechanisms of degradation in the body, “biodegradation” (hydrolysis etc.) may be different than the mechanisms of degradation during processing. Polymer degradation during the manufacturing may impact the mechanical properties of the final product.

### SUMMARY OF THE INVENTION

Various embodiments of the present invention include a bioabsorable stent. The stent body includes a may be fabricated from a biodegradable polyester, and at least one stabilizer. The stabilizer inhibits the degradation of the polyester during fabrication, and the stabilizer is selected from the group consisting of free radical scavengers, peroxide decomposers, catalyst deactivators, water scavengers, and metal scavengers.

Various embodiments of the present invention include a bioabsorable implantable medical device. The device includes a device body fabricated from a biodegradable polyester, and two or more stabilizers. At least two of the two or more stabilizers are of different categories and inhibit the degradation of the polyester during fabrication. The categories are selected from the group consisting of free radical scavengers, peroxide decomposers, catalyst deactivators, water scavengers, and metal scavengers.

Various embodiments of the present invention include a method of fabricating an implantable medical device. The method includes the operations of: forming an implantable medical device with at least two processing operations, the device body composed of a biodegradable polyester; adding at least one stabilizer during and/or prior to at least one of the processing operations wherein the stabilizer reduces or inhibits polymer degradation during at least one of the processing operations; and wherein the stabilizer is selected from the group consisting of free radical scavengers, peroxide decomposers, catalyst deactivators, water scavengers, and metal scavengers.

Various embodiments of the present invention include a method of fabricating an implantable medical device. The method includes the operations of: forming an implantable medical device with at least two processing operations, the device body composed of a biodegradable polyester; adding two or more stabilizers during and/or prior to any of the at least two processing operations wherein at least two of the two or more stabilizers reduce or inhibit polymer degradation during at least one of the processing operations. The at least two stabilizers are independently selected from the group consisting of free radical scavengers, peroxide decomposers, catalyst deactivators, water scavengers, and metal scavengers.

Various embodiments of the present invention include a method of fabricating a stent. The method includes the following processing operations: forming a polymeric tube utilizing extrusion, the polymer tube being formed from a biodegradable polyester; adding a stabilizer during extrusion; radially deforming the formed tube; cutting a stent pattern into the tube to form a stent; and sterilizing the stent. The stabilizer reduces or inhibits polymer degradation during at least one of the processing operations.

Various embodiments of the present invention include a method of fabricating a stent. The method of includes the following processing operations: adding a stabilizer to a biodegradable polyester; forming a polymeric tube utilizing extrusion, the polymer tube being formed from the biodegradable polyester; radially deforming the formed tube; cutting a stent pattern into the tube to form a stent; and sterilizing the stent. The stabilizer reduces or inhibits reduces or inhibits polymer degradation during at least one of the processing operations.

### BRIEF DESCRIPTION OF THE DRAWINGS

FIG. 1 depicts a stent.

### DETAILED DESCRIPTION OF THE INVENTION

Use of the singular herein includes the plural and vice versa unless expressly stated to be otherwise. That is, “a” and “the” refer to one or more of whatever the word modifies. For example, “a drug” includes one drug, two drugs, etc. Likewise, “the stabilizer” may refer to one, two or more stabilizers and “the polymer” may mean one polymer or a plurality of polymers. By the same token, words such as, without limitation, “stabilizers” and “polymers” would refer to one layer or polymer as well as to a plurality of layers or polymers unless, again, it is expressly stated or obvious from the context that such is not intended.

As used herein, unless specifically defined otherwise, any words of approximation such as without limitation, “about,” “essentially,” “substantially” and the like mean that the element so modified need not be exactly what is described but can vary from the description by as much as ±15% without exceeding the scope of this invention.

As used herein, “optional” means that the element modified by the term may or may not be present.

The various embodiments of the present invention include implantable medical devices, such as stents, manufactured from polymers, more particularly, biodegradable polymers such as, without limitation, biodegradable polyesters, polyanhydrides, or poly(ether-esters). The polymer may be a biostable polymer, a biodegradable polymer, or a blend of a biostable polymer and a biodegradable polymer. As noted above, processing of a polymer, such as, without limitation, poly(L-lactide) (PLLA), results in the polymer being exposed to elevated temperatures, moisture, viscous shear, and other potential sources of degradation, such as metals and metal catalysts. Certain embodiments of the present invention involve the addition of one or more stabilizers to the polymer before and/or during the manufacturing process to reduce or inhibit the degradation of the polymer that occurs during the processing, especially the decrease in polymer molecular weight.

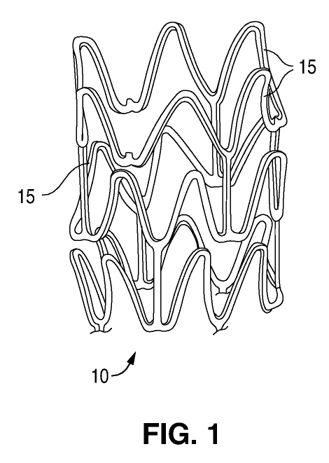

A stent may include a pattern or network of interconnecting structural elements or struts. FIG. 1 depicts an example of a three-dimensional view of a stent 10. The stent may have a pattern that includes a number of interconnecting elements or struts 15. The embodiments disclosed herein are not limited to stents or to the stent pattern illustrated in FIG. 1.

Although the discussion that follows focuses on a stent as an example of an implantable medical device, the embodiments described herein are easily applicable to other implantable medical devices, including, but not limited to self-expandable stents, balloon-expandable stents, stent-grafts, and grafts. The embodiments described herein are easily applicable to patterns other than that depicted in FIG. 1. The structural pattern of the device can be of virtually any design. The variations in the structure of patterns are virtually unlimited. The embodiments described herein are applicable to all polymers, including biodegradable polymers, biodegradable polyanhydrides, poly(ether-esters), or polyesters such as poly(L-lactide), poly (D,L-lactide), poly(L-lactide-co-D,L-lactide), poly(L-lactide-co-glycolide), poly(D,L-lactide-co-glycolide), poly(L-lactide-co-caprolactone), poly(glycolide-co-caprolactone), poly(D,L-lactide-co-caprolactone) and blends of the aforementioned polymers.

A stent such as stent 10 may be fabricated from a polymeric tube or a sheet by rolling and bonding the sheet to form the tube. A tube or sheet can be formed by extrusion or injection molding. A stent pattern, such as the one pictured in FIG. 1, can be formed in a tube or sheet with a technique such as laser cutting, machining or chemical etching. The stent can then be crimped on to a balloon or catheter for delivery into a bodily lumen.

The elevated temperatures, exposure to shear, exposure to moisture and exposure to radiation that is encountered in polymer processing may lead to degradation of the polymer. Such degradation may lead to a decrease in polymer molecular weight. In addition, polymer degradation can result in formation of oligomers, cyclic dimers, and monomers, with or without a significant decrease in molecular weight, which can alter the polymer properties and degradation behavior.

Some of the process operations involved in fabricating a stent may include:

(1) forming a polymeric tube using extrusion;

(2) radially deforming the formed tube by application of heat and/or pressure;

(3) forming a stent from the deformed tube by cutting a stent pattern in the deformed tube;

(4) coating the stent with a coating including an active agent;

(5) crimping the stent on a support element, such as a balloon on a delivery catheter;

(6) packaging the crimped stent/catheter assembly; and

(7) sterilizing the stent assembly.

The initial step in the manufacture of a stent is to obtain a polymer tube or sheet. The polymer tube or sheet may be formed using various types of forming methods, including, but not limited to, extrusion or injection molding. A polymer sheet may be rolled and bonded to form a polymer tube. Representative examples of extruders include, but are not limited to, single screw extruders, intermeshing co-rotating and counter-rotating twin-screw extruders and other multiple screw masticating extruders.

Both extrusion and injection molding expose the polymer to elevated temperatures and shear. In extrusion, a polymer melt is conveyed through an extruder and forced through a die as a film in the shape of a tube. Depending upon the type of extrusion and the molecular weight of the polymer, the polymer may be close to, at, or above its melting point. Specifically, the melt viscosity is desirably in a particular range to facilitate the extrusion process. In general, as the molecular weight increases, higher processing temperatures may be needed to achieve a melt viscosity that allows for processing. For example, for a biodegradable polyester such as poly(L-lactide), the temperature range may be in the range of about 180° C. to 220° C. for a melt extrusion operation. The residence time in the extruder may be about 5 minutes to about 30 minutes. These high temperatures, combined with the shear, moisture, residual catalyst, and other metals to which the polymer is exposed during extrusion, may lead to polymer degradation. The film can be cooled below the melting point, Tₘ, of the polymer to form an extruded polymeric tube. Alternatives to melt extrusion include gel extrusion, as well as extrusion using a supercritical fluid, or near supercritical fluid, such as without limitation, carbon dioxide near or above its critical point.

Upon exiting the extruder, the film in the shape of a tube can be axially drawn or stretched. As the tube is drawn, its diameter decreases. The tube may be cooled during expansion and/or after drawing.

Radial deformation of the formed tube is another processing step which may potentially cause degradation. Generally, application of strain can increase strength and modulus along the direction of strain. Thus, the formed film may be expanded in the radial direction to improve the radial strength of the polymer tube, and thus the stent formed from the deformed tube. The application of strain can induce molecular orientation along the direction of strain which can increase the strength and modulus along that direction. The tube can also be axially deformed to increase strength in the axial direction. The radial deformation is facilitated by an increase in temperature.

A technique for the radial deformation of a tube is blow molding. The polymeric tube is placed in a mold, and deformed in the radial direction by application of a pressure from a fluid. The pressure expands the tube such that it contacts the walls of the mold. The mold may act to limit the radial deformation of the polymeric tube to a particular diameter, the inside diameter of mold.

During the blow molding, the polymer tube may be heated by a heated gas or fluid, or the mold may be heated, thus heating the polymer tube within. After the tube has been blow molded to a particular diameter, the tube can be maintained under the elevated pressure and temperature for a period of time. The period of time may be between about one minute and about one hour, or more narrowly, between about two minutes and about ten minutes. This is referred to as “heat setting.”

As polymer chains have greater mobility above T_{g}, maintaining the polymer tube in a deformed state at a temperature above the T_{g}, that is heat setting the tube, allows the chains to rearrange closer to a thermodynamically equilibrium condition. Also, for polymers that are capable of crystallization, crystallization occurs at temperatures between the glass transition temperature and the melting temperature.

Thus, during radial expansion the film may be at a temperature between the glass transition temperature and the melting temperature. After expansion, the film may remain in the mold for a period of time at the elevated temperature of expansion. As an example, the polymer may be exposed to a temperature of about 80° C. to 160° C. for the duration of processing, about 3-15 minutes, and optionally heat set afterwards.

Once the polymeric tube has been formed, and optionally radially expanded, a stent pattern is cut into the tube. The stent pattern may be formed by any number of methods including chemical etching, machining, and laser cutting. Laser cutting generally results in a heat affected zone (HAZ). A HAZ refers to a portion of a target substrate that is not removed, but is still exposed to energy from the laser beam, either directly or indirectly. Direct exposure may be due to exposure to the substrate from a section of the beam with an intensity that is not great enough to remove substrate material through either a thermal or nonthermal mechanism. A substrate can also be exposed to energy indirectly due to thermal conduction and scattered radiation. The exposure to increased temperature in a HAZ may lead to polymer degradation.

In some embodiments, the extent of a HAZ may be decreased by the use of an ultrashort-pulse laser. This is primarily due to the increase in laser intensity associated with the ultrashort pulse. The increased intensity results in greater local absorption. “Ultrashort-pulse lasers” refer to lasers having pulses with durations shorter than about a picosecond (=10⁻¹²), and includes both picosecond and femtosecond (=10⁻¹⁵) lasers. Other embodiments include laser machining a stent pattern with a conventional continuous wave or long-pulse laser (nanosecond (10⁻⁹) laser) which has significantly longer pulses than utlrashort pulse lasers. There is a larger HAZ for a continuous or long-pulse laser as compared to an ultrashort pulse laser, and therefore the extent of polymer degradation is higher.

The stent formed from cutting the stent pattern into the polymeric tube may optionally be coated. The coating may be polymeric or non-polymeric and may optionally include an active agent. A coating material composed of a coating polymer dissolved in an organic solvent and optionally an active agent dispersed or dissolve in the solvent is generally applied at ambient, about 20° C. to about 25° C. After a coating material is applied, solvent is removed by blowing a warm gas on the stent for about 10 to 45 seconds, the temperature of the gas being roughly in the range of about 35° C. to about 45° C. About 5 to 20 passes by the spray coater and blow dryer may be required to obtain the desired coating layer thickness. If an active agent is included in the coating, the temperature stability of the active agent may be the limiting factor in choosing the temperature of the coating operation.

Subsequent to the coating operation, the stent may be exposed to an elevated temperature for some time to remove residual solvent. For example, the stent may be held at a temperature in the range of about 40° C. to about 80° C., for 30 minutes to 180 minutes.

Further embodiments can include fabricating a stent delivery device by crimping the stent on a support element, such as a catheter balloon, such that the temperature of the stent during crimping is above an ambient temperature. Heating a stent during crimping can reduce or eliminate radially outward recoiling of a crimped stent which can result in an unacceptable profile for delivery. Crimping may also occur at an ambient temperature. Thus, crimping may occur at a temperature ranging from 30° C. to 60° C. for a duration ranging from about 60 seconds to about 5 minutes.

Once the stent has been crimped onto a support element, such as without limitation, a catheter balloon, the stent delivery device is packaged and then sterilized. Ethylene oxide sterilization, or irradiation, either gamma irradiation or electron beam irradiation (e-beam irradiation), are typically used for terminal sterilization of medical devices. For ethylene oxide sterilization, the medical device is exposed to liquid or gas ethylene oxide that sterilizes through an alkalization reaction that prevents organisms from reproducing. Ethylene oxide penetrates the device, and then the device is aerated to assure very low residual levels of ethylene oxide because it is highly toxic. Thus, the ethylene oxide sterilization is often performed at elevated temperatures to speed up the process. Moisture is also added as it increases the effectiveness of ethylene oxide in eliminating microorganisms. Polymer degradation may occur due to the ethylene oxide itself interacting chemically with the polymer, as well as result from higher temperatures and the plasticization of the polymer resulting from absorption of ethylene oxide. More importantly, polymer degradation can occur from the combination of heat and moisture.

Alternatively, irradiation may be used for terminal sterilization. It is known that radiation can alter the properties of the polymers being treated by the radiation. High-energy radiation tends to produce ionization and excitation in polymer molecules. These energy-rich species undergo dissociation, subtraction, and addition reactions in a sequence leading to chemical stability. The degradation process can occur during, immediately after, or even days, weeks, or months after irradiation which often results in physical and chemical cross-linking or chain scission. Resultant physical changes can include embrittlement, discoloration, odor generation, stiffening, and softening, among others.

In particular, the deterioration of the performance of polymers due to e-beam radiation sterilization has been associated with free radical formation during radiation exposure and by reaction with other parts of the polymer chains. The reaction is dependent on e-beam dose, temperature, and atmosphere present. Additionally, exposure to radiation, such as e-beam, can cause a rise in temperature of an irradiated polymer sample. The rise in temperature is dependent on the level of exposure. In particular, the effect of radiation on mechanical properties may become more pronounced as the temperature approaches and surpasses the glass transition temperature, T_{g}. The deterioration of mechanical properties may result from the effect of the temperature on polymer morphology, but also from increased degradation resulting in a decrease in molecular weight. As noted above, degradation may increase above the glass transition temperature due to the greater polymer chain mobility.

Thus, in some embodiments sterilization by irradiation, such as with an electron beam, may be performed at a temperature below ambient temperature. As an example, without limitation, sterilization may occur at a temperature in the range of about −30° C. to about 0° C. Alternatively, the stent may be cooled to a temperature in the range of about −30° C. to about 0° C., and then sterilized by e-beam irradiation. The sterilization may occur in multiple passes through the electron beam. In other embodiments, sterilization by irradiation, such as with an electron beam, may occur at ambient temperature.

As outlined above, the manufacturing process results in the polymer's exposure to high temperatures and other potential sources of degradation, such as without limitation, irradiation, moisture, and exposure to solvents. In addition, residual catalysts in the polymer raw material, and other metals, such as from processing equipment, may catalyze degradation reactions. The polymer is also exposed to shear stress, particularly during extrusion. Thus, there are a number of sources of potential polymer degradation.

Polymer molecular weight may significantly decrease during the processing operations used in the manufacture of a stent. A non-limiting example is the use of a PLLA polymer to manufacture a stent. The stent manufacturing process involves extruding a polymer tube, radially expanding the polymer tube, laser cutting a stent pattern into the tube to form a stent, crimping the stent onto a balloon catheter, and sterilizing the crimped stent. The entire process results in a decrease of the weight average molecular weight from about 550 kg/mol to about 190 kg/mol. Extrusion of the polymer tube results in a decreases to about 380 Kg/mol from the initial 550 kg/mol. The molecular weight is further decreased to about 280 kg/mol after radial expansion and laser cutting. After sterilization by electron beam irradiation (25 KGy), the molecular weight (weight average) is about 190 kg/mol.

In general, the decomposition of a polymer, for example a biodegradable polyester such as, without limitation, PLLA, is due to exposure to heat, light, radiation, moisture, or other factors. As a result, a series of byproducts such as lactide monomers, cyclic oligomers and shorter polymer chains appear once the formed free radicals attack the polymer chain. In addition, decomposition may be catalyzed by the presence of oxygen, water, or residual metal such as from a catalyst. More specifically the polyester poly(L-lactide) is subject to thermal degradation at elevated temperatures, with significant degradation (measured as weight loss) occurring at about 150° C. and higher temperatures. The polymer is subject to random chain scission. To explain the presence of lactide at higher temperatures, some have postulated the existence of an equilibrium between the lactide monomer and the polymer chain. In addition to lactide, the degradation products also include aldehydes, and other cyclic oligomers. Although the degradation mechanisms of PLLA are not fully understood, a free radical chain process can be involved in the degradation. Other mechanisms include depolymerization due to attack by the hydroxyl groups at the chain ends, ester hydrolysis occurring anywhere on the polymer due to water, and thermally driven depolymerization occurring anywhere along the polymer chain. In the cases of depolymerization occurring by backbiting from the terminal hydroxyl groups or thermally driven along the polymer backbone, these process may be especially accelerated by the presence of polymerization catalysts, metal ions, and Lewis acid species.

Various embodiments of the present invention involve the addition of one or more stabilizers to the polymer before and/or during the processing to reduce or inhibit polymer degradation during the manufacture of the implantable medical device, or stent, and especially to reduce or inhibit the decrease in the polymer molecular weight.

One category of stabilizers is free radical scavengers. These are also sometimes referred to as antioxidants. “Free radicals” refer to atomic or molecular species with unpaired electrons on an otherwise open shell configuration. Free radicals can be formed by oxidation reactions. These unpaired electrons are usually highly reactive, so radicals are likely to take part in chemical reactions, including chain reactions. Free radical scavengers operate through donation of an electron or hydrogen to a free radical, thus removing the free radical from further reaction. The free radical scavenger effectively competes with the polymer for the free radicals, and thus removes the free radicals from the reaction cycle.

Some representative examples of free radical scavengers include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N,N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, melatonin, carotenes, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentaerythritol diphosphite, tris(2,4-di-tert.butyl phenyl)phosphite, dilauryl thiodipropionate and bis(2,4-di-tert.butyl phenyl)pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl)propionate methane 2,5-di-tert-butylhydroquinone, ionol, pyrogallol, retinol, and octadecyl-3-(3,5-di-tert.butyl-4-hydroxyphenyl)propionate.

Other free radical scavengers, such as various isomers of Vitamin E, may be used, including the four tocopherols and four tocotrienols. The alpha, beta, gamma and delta forms of both the tocopherols and tocotrienols may be used to prevent chemical degradation.

In a biodegradable implant, any antioxidant would ultimately be released. Hence, antioxidants which are food grade or which are biocompatible are preferred. These preferred antioxidants would include BHT, BHA, trihydroxybutyrophenone, L-ascorbic acid, (Vitamin C), sodium ascorbate, Vitamin E, herbal rosemary, sage extracts, glutathione, melatonin, carotenes, carotenoids, resveratrol, methyl gallate, n-octyl gallate, n-dodecyl gallate, propyl gallate, propyl paraben, luteolin, eriodictyol, astaxanthin, anthocyanins, carnosol, quercetin, ethoxyquin, catechin, morin, rutin, boldine, tocopherols, hydroxytyrosol, ubiquinol, isoflavones, lycopene, fisetin, ellagic acid, L-DOPA, sinapine, olivetol, dehydrozingerone, curcumin, and tertbutylhydroquinone.

Another category of stabilizers is peroxide decomposers. Peroxide decomposers act by removing an oxidative catalyst present in polymer resins, which is a hydroperoxide, or peroxide. Hydroperoxides readily decompose to create free radicals. Peroxide decomposers react with hydroperoxides to create non-free radical species, and thus help inhibit oxidation. Examples include trivalent phosphorous and divalent sulfur compounds such as sulfites, thiodipropionates and organophosphites. Other examples of peroxide decomposers are esters of β-thiodipropionic acid, such as without limitation, for example the lauryl, stearyl, myristyl or tridecyl ester, and salts of 2-mercaptobenzimidazole, for example the zinc salt, and diphenylthiourea. Among the more stable trivalent phosphorous compounds are dicumylphosphite, tris(2,4 di-tert-butylphenyl)phosphate, and tetrakis(2,4-di-tert-butylphenyl) 4,4′-biphenylenediphosphonite. Also, hydroxylamines are both free-radical scavengers and decompose hydroperoxides.

Another category of stabilizers are catalyst deactivating agents. These agents reduce the catalytic decomposition of the polymer resulting from residual metal in polymer resins, and may also be referred to as “metal deactivators.” In general, these compounds complex with the metal ion or the catalytic metal ion complex, such as stannous octoate, so that the metal can no longer act as a catalyst for polymerization or depolymerization. Non-limiting examples of catalyst-deactivating agents include hindered, alkyl, aryl and phenolic hydrazides, amides of aliphatic and aromatic mono- and dicarboxylic acids, cyclic amides, hydrazones and bishydrazones of aliphatic and aromatic aldehydes, hydrazides of aliphatic and aromatic mono- and dicarboxylic acids, bis-acylated hydrazine derivatives, and heterocyclic compounds. Other compounds include isopropanolamines, phosphate esters, tri-sodium phosphate, tri-potassium phosphate, alkyl or aromatic amines, amides, L-DOPA, dopamine, 1,4-diaminobutane, 1,5-diaminopentane, glutathione, and alkoxides. A non-limiting example of a specific compound is 1,2-bis(3,5-di-tert-butyl-4-hydroxyhydro cinnamoyl)hydrazine (BNX® MD-1024 from Mayzo or IRGANOX MD 1024 from Ciba-Geigy).

Another category of stabilizers are water or moisture scavengers. All biodegradable polyesters, such as PLLA, are susceptible to water induced hydrolytic degradation, which is not surprising as this is a primary degradation mechanism in vivo. Water, combined with a catalyst can be particularly effective at hydrolyzing biodegradable polyesters. Suitable water scavengers are alkoxy silanes, anhydrides, carbodiimides, isocyanates, aluminosilicates, zeolites, alumina, silica, calcium chloride, calcium carbonate, potassium carbonate, carbonates, sodium sulfate, magnesium sulfate, calcium sulfate. Many of these inorganic compounds would be present as a discrete particle, particulate, or nanoparticles in the polyester resin. For optimal properties, these materials would need to have a small particles size, less than 10 microns, and more optimally, less than one micron. Many of these inorganic dry agents, such as sodium sulfate and calcium chloride, would dissolve upon release and be quite biocompatible.

A final category of stabilizers is metal scavengers which includes both chelating agents and cryptands. Cryptands are a “family of synthetic bi- and polycyclic multidenate ligands for a variety of cations.” Cryptands bind cations using both oxygen and nitrogen atoms. Metal chelators and cryptands scavenge and tie up residual metal to prevent the metal from associating with a hydroperoxide which is required to catalyze the depolymerization. Some non-limiting examples of chelating agents are ethylene diamine tetraacetic acid (EDTA), diethylene triamine pentaacetic acid (DPTA), nitrilotriacetic acid (NTA) porphyrin rings, histidine, malate, phytochelatin, humic acid, and oxalic acid. A non-limiting example of a cryptand is N[CH₂CH₂OCH₂CH₂OCH₂CH₂]₃N.

Any type of combination of the above mentioned stabilizers may be used in the various embodiments of the present invention.

Some of the stabilizers utilized in the various embodiments of the present invention are compounds which are also used therapeutically. The stabilizers discussed in the various embodiments of the present invention are intended to inhibit the degradation of the polymer backbone of the implantable medical device, such as a stent. In some embodiments, a compound which may be used therapeutically and thus may be categorized as an antioxidant due to its therapeutic effects may be added to the process to inhibit degradation of the polymer. In other embodiments, compounds categorized as antioxidants and which may also have a therapeutic effect may be specifically excluded.

Various embodiments of the present invention include methods of fabricating an implantable medical device wherein the device body is formed from a polymer in which one or more stabilizers has been added to the polymer before and/or during processing. In the various embodiments, the polymer may be a biostable polymer, a biodegradable polymer such as, without limitation, a biodegradable polyester, or a blend of biostable and biodegradable polymers. In some embodiments, a combination of a biostable and a biodegradable polymer may be used. As used herein, “use of a stabilizer” means that the stabilizer is added to the polymer raw material prior to processing and/or is added to the polymer or polymer formulation during the processing of the implantable medical device. As used herein, a “polymer formulation” is a composition including a polymer as a major component, but also may include fillers, particles, plasticizers, and/or other materials. The stabilizers are added to inhibit or reduce the degradation of the polymer during processing including reducing or inhibiting the decrease of polymer molecular weight. The various embodiments are discussed in the following paragraphs.

As used herein when an implantable medical device, such as a stent, is said to be fabricated from a polymer, or the device or device body is composed of a polymer, it means the body of the device is made from a polymer or a polymer formulation. Thus, for a stent which is “fabricated from a biodegradable polyester,” or “composed of a biodegradable polyester,” the body of the stent may be completely, or substantially completely, a biodegradable polyester. The body of the stent may be made from a composition including a polyester and other materials, such that the polyester is the continuous phase. The body of the stent may be at least 50% by weight biodegradable polyester. In other embodiments, biodegradable polyester may be at least 50% by volume of the composition forming the stent body. Similarly, a tube referred to as a polymeric tube may be formed from a polymer or a polymer formulation.

In some embodiments, the fabrication of the implantable medical device may include at least one melt processing operation, while others may include at least two operations where the processing temperature is above the glass transition temperature of the polymer. In some embodiments, the fabrication of the implantable medical device may include at least one melt processing operation and at least one additional operation where the processing temperature is above the glass transition temperature of the polymer. The various processing operations may occur at a temperature of at least 160° C., at least 180° C., at least 200° C., or at least 210° C.

In some embodiments, the fabrication of the implantable medical device may include any of the processing operations previously discussed above. These processing operations include forming a polymeric tube using extrusion, radially deforming the formed tube, forming a stent from the deformed tube, crimping the stent, and sterilizing the stent wherein the order of the steps is as presented except that sterilization could be carried out at any earlier point in the process. The various embodiments encompass all of the variations in the processing operations discussed above.

In the various embodiments of the present invention, the concentrations of the stabilizer may vary from about 0.001% weight percent up to about 5% weight percent, or more narrowly 0.01% to 2% weight percent. The weight percent for the stabilizer refers to the weight percent with respect to the polymer, and not the polymer formulation as a whole. In other words 0.001% is one part by weight stabilizer to 1000 parts by weight of the sum of stabilizer and polymer (or in other words 0.001% is 1 part by weight stabilizer to 999 parts by weight polymer). Thus, non polymer components are not included in the calculation of weight percent of stabilizer.

Various embodiments of the present invention include the addition of one or more stabilizers to the polymer or the polymer formulation. The stabilizers are selected from the categories described above, that is, free radical scavengers, peroxide decomposers, catalyst deactivators, water scavengers, and metal scavengers. In some embodiments, the stabilizer may be added to the polymer resin, which is the polymer raw material, prior to any processing. In some embodiments, the stabilizer may be added to the polymer or polymer formulation in the extruder, or during the first processing operation. In other embodiments, the stabilizer may be added during more than one processing operation, and/or different stabilizers may be added at different points during the processing. In some embodiments, one or more stabilizers may be added prior to any processing, and/or one or more stabilizers may be added during the processing. In some embodiments, the different stabilizers from the different categories may be added at different points in the process. The stabilizers can be added either to the polymer prior to addition to the extruder, added to the extruder and/or at multiple points during the extrusion, or any combination thereof.

In some embodiments, only one stabilizer may be used, but the stabilizer may not be added to the polymer all at once. That is, some of the stabilizer may be added prior to the processing, or at a particular point in the processing, and the remaining stabilizer may be added at different points during the processing. In some embodiments, the same stabilizer may be added at two or more times points in the processing where prior to processing may be one of the points of addition.

In those embodiments in which the stabilizer is added to the raw material, that is the resin or pellets of polymer, prior to any processing, there are a number of ways to accomplish the addition. One method is to add the stabilizer as a dry powder, to the polymer raw material, often available as pellets, and blend these together in a mixer such as twin-cone blender, tumbler, V-blender or the like. In some embodiments, such a mixer may also be used for the addition of a liquid phase stabilizer to the polymer. Due to the low concentration of stabilizer, and the need for a reasonably uniform distribution of the stabilizer, geometric blending may be used. Geometric blending involves first making a concentrated pre-blend of the stabilizer and polymer (or polymer formulation), and then successively diluting this blend with additional polymer (or polymer formulation). As a non-limiting example, a 1:8 mass basis of stabilizer to polymer may be made, and then this blend successively diluted by the addition of more polymer at a 1:1 or 1:2 ratio or the like, until all of the polymer has been blended. The geometric blending could be accomplished using any of the mixers outlined above. In other embodiments, the concentrated blend of stabilizer and polymer may be added to the extruder which includes the polymer or polymer formulation.

Other embodiments include forming a concentrated blend of polymer and stabilizer by dissolving both in a solvent, and then either precipitating the polymer and stabilizer from the solvent, or alternatively, removing the solvent by evaporation. A concentrated pre-blend of the polymer and stabilizer would then result. In other embodiments, a concentrated pre-blend may be obtained by dissolving or dispersing the stabilizer in a solvent, and then spraying the solution onto the polymer or polymer pellets in equipment such as a tablet coater, or a fluid-bed processor/granulator with a Wurster insert. In either case, the concentrated preblend may be either geometrically blended with the other polymer or polymer formulation, or alternatively added to the extruder to mix the concentrated blend with the other material, or blended by some other means.

Various embodiments of the present invention include the use of one stabilizer in the processing of the polymer. The stabilizer may belong to one of the following categories of stabilizers: free radical scavengers, peroxide decomposers, catalyst deactivators, water scavengers, or metal scavengers. In some embodiments, the stabilizer may be a catalyst deactivator, such as without limitation, 1,2-bis(3,5-di-tert-butyl-4-hydroxyhydro cinnamoyl)hydrazine.

Various embodiments of the present invention include the use of at least two stabilizers, each of which is chosen from a separate category and which are not the same material. As there are potentially multiple degradation mechanisms, and multiple methods of reducing or inhibiting degradation, it may be advantageous to use more than one type of stabilizer. Thus, some embodiments include use of a stabilizer from each of the categories (five or more stabilizers), while other embodiments include the use of stabilizers from four of the five categories (four or more stabilizers). Further, some embodiments include use of stabilizers from three of the five categories (three or more stabilizers). Some embodiments include the use of more than one stabilizer from the same category, either with or without one or more stabilizers from another category.

Other stabilizers that do not fall into one the above specifically enumerated categories may be used with any of the embodiments of the present invention.

The stabilizers added to the polymer, such as a biodegradable polyester like PLLA, must be acceptable for use in an implantable medical device, and the byproducts must also be acceptable for use in an implantable medical device. Specific preferred antioxidants include BHT, BHA, trihydroxybutyrophenone, L-ascorbic acid, (Vitamin C), sodium ascorbate, Vitamin E, herbal rosemary, sage extracts, glutathione, melatonin, carotenes, carotenoids, resveratrol, methyl gallate, n-octyl gallate, n-dodecyl gallate, propyl gallate, propyl paraben, luteolin, eriodictyol, ethoxyquin, astaxanthin, anthocyanins, carnosol, quercetin, catechin, morin, rutin, boldine, tocopherols, hydroxytyrosol, ubiquinol, isoflavones, lycopene, fisetin, ellagic acid, L-DOPA, sinapine, olivetol, dehydrozingerone, curcumin, and tertbutylhydroquinone.

In the category of peroxide decomposers, preferred compounds for biocompatibility are sulfites, thiodipropionates, β-thiodipropionic acid, such as without limitation, for example the lauryl, stearyl, myristyl or tridecyl ester.

In the category of catalyst deactivating agents, preferred compounds for biocompatibility are amides of aliphatic and aromatic mono- and dicarboxylic acids, cyclic amides, phosphate esters, tri-sodium phosphate, tri-potassium phosphate, L-DOPA, dopamine, 1,4-diaminobutane, 1,5-diaminopentane, and glutathione.

In the category of water scavengers, preferred compounds for biocompatibility are potassium carbonate, carbonates, sodium sulfate, magnesium sulfate, calcium sulfate, calcium chloride, and calcium carbonate. If they are used in nanoparticulate form (<300 nm size) then nanoparticles of aluminosilicates, zeolites, alumina, silica are also possible.

In the category of metal scavengers, preferred compounds for biocompatibility are ethylene diamine tetraacetic acid (EDTA), porphyrin rings, histidine, malate, phytochelatin, and salts of oxalic acid.

Among other factors to consider in choosing the one or more stabilizers is the temperature stability of the stabilizer. Thus, for processing operations that occur at elevated temperatures, the stabilizer may not be so volatile that it cannot reduce the extent of polymer degradation during polymer processing. In addition, it is desired that the stabilizer not be so thermally unstable so as to not persist after polymer processing. Another consideration in the case of multiple stabilizers is the compatibility of the stabilizers.

Some stabilizers are especially suited for melt processing operations. These stabilizers include hindered phenols, phosphites, hydroxylamines, and α-tocopherol. These compounds are stable at higher temperatures encountered in melt-processing, and in some cases, are more effective at the temperatures encountered in melt processing. Particular combinations that may be used in the various embodiments of the present invention include a phenolic antioxidant and a phosphite or a phenolic antioxidant and a thioester.

Some embodiments of the present invention include other stabilizer combinations. Another useful combination is a free radical scavenger and a peroxide decomposer. Some specific examples include dialkyl thiodipropionates and hindered phenols in combination which give a synergistic effect at high temperatures. Another non-limiting example is the use of trivalant phosphorous compounds and hindered phenols in combination.

Specific preferred combinations include a catalyst deactivator and at least one other stabilizer that is selected from one of the categories of free radical scavengers, peroxide decomposers, water scavengers, or metal scavengers. An exemplary catalyst deactivator is 1,2-bis(3,5-di-tert-butyl-4-hydroxyhydro cinnamoyl)hydrazine (IRGANOX® MD 1024 from Ciba-Geigy Corporation). A preferred combination of a catalyst deactivator and an antioxidant is dopamine and propyl gallate.

Another preferred combination is an antioxidant such as BHT, propyl gallate, or trihydroxybutyrophenone and a water scavenger such as potassium carbonate or calcium sulfate. Yet another combination is the peroxide decomposer dilauryl thiodiproprionate and the metal scavengers EDTA or sodium oxalate. Another preferred combination is the catalyst deactivator n-methyl pyrrolidone combined with the antioxidant BHT.

The resulting implantable medical device, such as a stent, fabricated from a polymer such as a biodegradable polyester, may include one or more stabilizers in the device body. In some embodiments, the stabilizers may be mixed or dispersed throughout the polymer, or polymer formulation, from which the device body has been fabricated, or substantially throughout the device body. In other embodiments, the stabilizers may be non-uniformly distributed. In still other embodiments, one or more stabilizers may be uniformly, or substantially uniformly, distributed throughout the polymer or polymer formulation from which the device body is fabricated, and one or more other stabilizers may be non-uniformly distributed.

In some embodiments, the implantable medical device may contain no or negligible quantities of the stabilizers added to the polymer due to consumption of the stabilizers during the processing. Thus, in some embodiments, the amount of the stabilizer present in the stent resulting from the fabrication with one or more stabilizers may be about 90% or less, about 80% or less, about 70% or less, about 60% or less, about 50% or less, about 40% or less, about 30% or less, about 20% or less, or about 10% or less of the total stabilizer added. In some embodiments, the stabilizer remaining in the implantable medical device after fabrication may be 5% or less, or even 2% or less, or 1% or less of the original stabilizer added. Different stabilizers may be consumed at different rates. Thus as a non-limiting example, there may be about 5% of the original quantity of one stabilizer present while a second stabilizer may be about 90% or more of the original quantity.

In some embodiments, the polymer of the device body processed with stabilizers may have a weight average molecular weight less than the weight average molecular weight of the polymer raw material. In such embodiments, the polymer has a weight average molecular weight of about 35% or more, about 40% or more, about 45% or more, about 50% or more, about 60% or more, about 65% or more, about 70% or more, about 75% or more, or about 80% or more of the original weight average molecular weight of the polymer raw material.

In some embodiments, the polymer of the device body processed with stabilizers may have a polydispersity greater than the polydispersity of the polymer raw material. In such embodiments, the polymer has a polydispersity (ratio of the polymer's weight average molecular weight to the polymer's number average molecular weight, or M_{w}/Mₙ) of not more than 2.2, or not more than 2.1, or not more than 2.0. In other embodiments, the resulting implantable medical device includes a polymer in the device body that has a polydispersity not more than 25%, or 20%, or 15%, or 10% greater than the polydispersity of the polymer raw material.

Note that the mechanical strength of a polymer, and thus an article fabricated from a polymer, is a function of the molecular weight. Thus, a drop in the molecular weight during processing decreases the mechanical strength.

### Polymers

Representative examples of polymers that may be used to fabricate an implantable medical device include, but are not limited to, poly(N-acetylglucosamine) (Chitin), Chitosan, polyesters, biodegradable polyesters, poly(hydroxyvalerate), poly(lactide-co-glycolide), poly(hydroxybutyrate), poly(hydroxybutyrate-co-valerate), polyorthoesters, polyanhydrides, poly(glycolic acid), poly(glycolide), poly(L-lactic acid), poly(L-lactide), poly(L-lactide-co-D,L-lactide), poly(D,L-lactic acid), poly(L-lactide-co-glycolide); poly(D,L-lactide), poly(D,L-lactide-co-glycolide), poly(L-lactide-co-caprolactone), poly(glycolide-co-trimethylene carbonate), poly(caprolactone), poly(trimethylene carbonate), polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

As used herein, the terms poly(D,L-lactide), poly(L-lactide), poly(D,L-lactide-co-glycolide), and poly(L-lactide-co-glycolide) are used interchangeably with the terms poly(D,L-lactic acid), poly(L-lactic acid), poly(D,L-lactic acid-co-glycolic acid), and poly(L-lactic acid-co-glycolic acid), respectively.

### Active Agents

Active agents, or drugs, may optionally be included either in the body of the implantable medical device such as a stent, and/or in a coating on the device. These active agents can be any agent which is a therapeutic, prophylactic, or a diagnostic agent, or any agent that is used to treat a disease or condition.

### Definitions

A “melt processing operation” refers to one in which the polymer, composition including a polymer, or other material, is processed at or above the melting temperature and the material is free of, or substantially free of, crystals or crystallites.

As used herein, a “processing operation temperature” refers to the temperature or temperature range utilized during a processing operation. The temperature during start-up time for a process, or temporary temperature excursions, are not “the processing operation temperature.”

As used herein, the “initial molecular weight of a polymer,” refers to the molecular weight, whether measured as a weight-average molecular weight, a number-average molecular weight, a viscosity average molecular weight, or other average molecular weight, of the material prior to any polymer processing operations.

As used herein, the terms “biologically degradable” (or “biodegradable”), “biologically erodable” (or “bioerodable”), “biologically absorbable” (or “bioabsorbable”), and “biologically resorbable” (or “bioresorbable”), in reference to polymers, coatings, or other materials referenced herein, are used interchangeably, and refer to polymers, coatings, and materials that are capable of being completely or substantially completely, degraded, dissolved, and/or eroded over time when exposed to physiological conditions, and can be gradually resorbed, absorbed and/or eliminated by the body, or that can be degraded into fragments that can pass through the kidney membrane of an animal (e.g., a human). Conversely, a “biostable” polymer, coating, or material, refers to a polymer, coating or material that is not biodegradable.

As used herein, “degradation” of a polymer refers to at least a decrease in the molecular weight of the polymer, and also encompasses other undesirable changes such as discoloration and oxidation, and/or the appearance of other chemical species.

Thus, a biodegradable polymer may “degrade” during polymer processing, and “biodegrade” when the polymer is implanted in the body. The mechanisms of degradation in the body, “biodegradation” (hydrolysis etc.) may be different than the mechanisms of processing degradation.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true spirit and scope of the embodiments of this invention.

What is claimed is:

1. A bioabsorable stent, the stent comprising:
a stent body fabricated from a biodegradable polyester;
the stent body including at least one stabilizer;
wherein the stabilizer inhibits the degradation of the polyester during fabrication; and
wherein the stabilizer is selected from the group consisting of free radical scavengers, peroxide decomposers, catalyst deactivators, water scavengers, and metal scavengers.

2. The stent of claim 1, wherein the stabilizer is homogeneously or substantially homogeneously mixed throughout the body of the stent.

3. The stent of claim 1, wherein the free radical scavenger is selected from BHT, BHA, hindered phenolics, propyl gallate, alkyl gallates, propyl paraben, luteolin, carnosol, catechin, quercetin, fisetin, olivetol, tocopherols, tertbutylhydroquinone, and trihydroxybutyrophenone.

4. The stent of claim 1, wherein the peroxide decomposer is an alkyl diester of thiodipropionic acid.

5. The stent of claim 1, wherein the catalyst deactivator is selected from N-methylpyrrolidone, 1,4-diaminobutane, 1,5-diaminopentane, glutathione, L-DOPA, dopamine, phosphate esters, trisodium phosphate, tripotassium phosphate, and 1,2-bis(3,5-di-tert-butyl-4-hydroxyhydro cinnamoyl)hydrazine.

6. The stent of claim 1, wherein the water scavenger is selected from sodium sulphate, calcium sulphate, magnesium sulphate, potassium carbonate, calcium chloride, alumino silicates, zeolites, alumina, and silica.

7. The stent of claim 1, wherein the metal scavenger is selected from EDTA and oxalate salts.

8. A bioabsorable implantable medical device, the device comprising:
a device body fabricated from a biodegradable polyester;
the device body comprising two or more stabilizers;
wherein at least two of the two or more stabilizers are of different categories and
inhibit the degradation of the polyester during fabrication; and
wherein the categories are selected from the group consisting of free radical scavengers, peroxide decomposers, catalyst deactivators, water scavengers, and metal scavengers.

9. The device of claim 8, wherein at least one of the at least two of the two or more stabilizers is homogeneously or substantially homogeneously mixed throughout the body of the stent.

10. The device of claim 8, wherein at least one of the two or more stabilizers is a catalyst deactivator.

11. The device of claim 10, wherein the catalyst deactivator is dopamine.

12. The device of claim 10, wherein the catalyst deactivator is 1,2-bis(3,5-di-tert-butyl-4-hydroxyhydro cinnamoyl)hydrazine.

13. A method of fabricating an implantable medical device, the method comprising:
forming an implantable medical device with at least two processing operations, the device body composed of a biodegradable polyester; and
adding at least one stabilizer during and/or prior to at least one of the processing operations wherein the stabilizer reduces or inhibits polymer degradation during at least one of the processing operations;
wherein the stabilizer is selected from the group consisting of free radical scavengers, peroxide decomposers, catalyst deactivators, water scavengers, and metal scavengers.

14. The method of claim 11, wherein one of the at least two processing operations is sterilization of the implantable medical device.

15. The method of claim 11, wherein one of the at least two processing operations is a melt processing operation in which the processing temperature is about 180° C. or greater.

16. The method of claim 11, wherein the at least two processing steps are selected from the group consisting of melt processing of the biodegradable polyester, extrusion of the biodegradable polyester, radial deformation of a polymer tube comprising the biodegradable polyester at a temperature greater than the polyester's glass transition temperature, laser machining a polymer tube comprising the biodegradable polyester, crimping the implantable medical device body, and sterilizing the implantable medical device body.

17. The method of claim 11, wherein the weight average molecular weight of the biodegradable polyester prior to any processing operations is the initial weight average molecular weight, and the biodegradable polyester in the fabricated implantable medical device has a weight average molecular weight of about 50% or greater than 50% of the initial weight average molecular weight of the biodegradable polyester.

18. The method of claim 11, wherein the weight average molecular weight of the biodegradable polyester prior to any processing operations is the initial weight average molecular weight, and the biodegradable polyester in the fabricated implantable medical device has a weight average molecular weight of about 60% or greater than 60% of the initial weight average molecular weight of the biodegradable polyester.

19. The method of claim 11, wherein the stabilizer is 1,2-bis(3,5-di-tert-butyl-4-hydroxyhydro cinnamoyl)hydrazine.

20. A method of fabricating an implantable medical device, the method comprising:
forming an implantable medical device with at least two processing operations, the device body composed of a biodegradable polyester; and
adding two or more stabilizers during and/or prior to any of the at least two processing operations wherein at least two of the two or more stabilizers reduce or inhibit polymer degradation during at least one of the processing operations;
wherein the at least two stabilizers are independently selected from the group consisting of free radical scavengers, peroxide decomposers, catalyst deactivators, water scavengers, and metal scavengers.

21. The method of claim 20, wherein free radical scavengers, peroxide decomposers, catalyst deactivators, water scavengers, and metal scavengers are the five categories of stabilizers, and wherein the at least two stabilizers are of different categories.

22. The method of claim 20, wherein one of the at least two processing operations is sterilization of the implantable medical device.

23. The method of claim 20, wherein one of the at least two processing operations is melt processing operation in which the processing temperature is about 180° C. or greater.

24. The method of claim 20, wherein the at least two processing steps are selected from the group consisting of melt processing of the biodegradable polyester, extrusion of the biodegradable polyester, radial deformation of a polymer tube comprising the biodegradable polyester at a temperature greater than the polyester's glass transition temperature, laser machining a polymer tube comprising the biodegradable polyester, crimping the implantable medical device body, and sterilizing the implantable medical device body.

25. The method of claim 20, wherein the weight average molecular weight of the biodegradable polyester prior to any processing operations is the initial weight average molecular weight and the biodegradable polyester in the fabricated implantable medical device has a weight average molecular weight of about 50% or greater than 50% of the initial weight average molecular weight of the biodegradable polyester.

26. The method of claim 20, wherein the weight average molecular weight of the biodegradable polyester prior to any processing operations is the initial weight average molecular weight and the biodegradable polyester in the fabricated implantable medical device has a weight average molecular weight of about 60% or greater than 60% of the initial weight average molecular weight of the biodegradable polyester.

27. The method of claim 20, wherein at least one of the two or more stabilizers is a catalyst deactivator.

28. The method of claim 27, wherein the catalyst deactivator is 1,2-bis(3,5-di-tert-butyl-4-hydroxyhydro cinnamoyl)hydrazine.

29. A method of fabricating a stent, the method comprising the following processing operations:
forming a polymeric tube utilizing extrusion, the polymer tube being formed from a biodegradable polyester;
adding a stabilizer during extrusion;
radially deforming the formed tube;
cutting a stent pattern into the tube to form a stent; and
sterilizing the stent;
wherein the stabilizer reduces or inhibits polymer degradation during at least one of the processing operations.

30. The method of claim 29, wherein radially deforming the polymeric tube is radially expanding the tube by blow-molding the polymeric tube.

31. The method of claim 29, wherein laser machining is used to cut a stent pattern in the tube to form a stent.

32. The method of claim 29, wherein the laser utilized in cutting a stent pattern is an ultrashort-pulse laser.

33. The method of claim 29, wherein electron beam irradiation is used to sterilize the stent.

34. The method of claim 29, further comprising coating the stent after the stent pattern is cut into the polymer tube to form the stent.

35. The method of claim 34, further comprising crimping the stent onto a support member after coating the stent and prior to sterilizing the stent.

36. A method of fabricating a stent, the method comprising the following processing operations:
adding a stabilizer to a biodegradable polyester;
forming a polymeric tube utilizing extrusion, the polymer tube being formed from the biodegradable polyester;
radially deforming the formed tube;
cutting a stent pattern into the tube to form a stent; and
sterilizing the stent;
wherein the stabilizer reduces or inhibits reduces or inhibits polymer degradation during at least one of the processing operations.
